(19) United States
(12) Patent Application Publication
(10) Pub. No.: US 2026/0130605 A1
(43) Pub. Date: May 14, 2026

(54) Modular Smart Body Fat Scale with Pet Body Configuration Detection Function

(71) Applicant: Mella Pet Care. Inc, Chicago, IL (US)

(72) Inventors: Anya Babbitt, Chicago, IL (US); Yue Zhang, Chicago, IL (US); Benjamin Seidman, Chicago, IL (US); Yi Wang, Chicago, IL (US)

(21) Appl. No.: 19/032,349

(22) Filed: Jan. 20, 2025

Related U.S. Application Data
(63) Continuation of application No. PCT/US2023/017768, filed on Apr. 6, 2023.

(30) Foreign Application Priority Data
Jul. 20, 2022 (CN) 202210859947.2

(51) Int. Cl. A61B 5/0537; A61B 5/00; A61B 5/107; G01G 17/08

(52) U.S. Cl. CPC A61B 5/0537; A61B 5/1075; A61B 5/1079; A61B 5/4872; G01G 17/08; A61B 2503/40

(57) ABSTRACT

The present disclosure discloses a modular smart body fat scale with a pet body configuration detection function, including a main body base and a wireless display. An installing cavity is formed in a front side of the top of the main body base, four weighting sensors arranged at equal intervals are disposed at the inside top of the installing cavity, an installing box is disposed at a top end of the weighting sensor, a matrix grid electrode is disposed inside the installing box, and a cover plate is disposed on the top of the installing box. A support frame bottom groove is formed in the back portion of the top of the main body base, a bottom motor box is disposed in the inside middle portion of the support frame bottom groove, a support upright post is disposed on a top end of the bottom motor box.

### CROSS-REFERENCE TO RELATED APPLICATION

This application is a bypass continuation application of PCT application no.: PCT/US2023/017768. This application claims priorities from PCT Application PCT/US2023/017768, filed Apr. 6, 2023, and from Chinese patent application 202210859947.2, filed Jul. 20, 2022, the contents of which are incorporated herein in the entirety by reference.

### TECHNICAL FIELD

The present disclosure relates to the technical field of a body fat scale, and more particularly relates to a modular smart body fat scale with a pet body configuration detection function.

### BACKGROUND

The trouble brought by overweight and obesity is not a problem only troubling modern humans, but is also a growing problem troubling pets. The Association for Pet Obesity Prevention estimated that more than 54 percent of pets in the United States are overweight or obese. Overweight and obesity will bring many adverse effects to the health of pets. Overweight will increase the load on joints, and make animals susceptible to bone and joint diseases. Overweight will also make the heart, lungs and other organs heavily burden, and increase the risk of heart diseases.

Under an overweight condition, the drug consumption calculated according to the body weight will also increase, adverse drug reactions are easier to occur, and the risk of anesthesia is especially greater. Too much fat makes surgery difficult. Besides, obesity is closely related to diabetes and some cancers. These are serious problems, but it is difficult to realize quantitative diagnosis on pet obesity.

A known method of measuring pet cats and dogs is BCS, which mainly adopts visual examination and palpation during evaluation, and the bone, subcutaneous fat, muscle, waist, and abdominal contours are examined emphatically. Then, a corresponding score is given according to a BCS score sheet. The visual examination and palpation have very high professional requirements on a veterinarian, much experience is needed, and errors are great since subjective judgments are given by people.

Another method of measuring the body fat in dogs is DEXA (Dual Energy X-ray Absorptiometry) analysis. This method requires dog anesthesia, and a 15-minute full body X-ray examination is needed to determine bone/body density. This method is invasive, time-consuming, and expensive. Although the results are very accurate, this method is not practical to use in general veterinary examination or cosmetic examination.

In recent years, bioelectrical impedance analysis (BIA) has been widely accepted as a method to measure the human body fat percentage. Body fat measurement by bioelectrical impedance is also applied to pets. However, the biological resistance takes a small effect during the current bioelectrical impedance measurement, in most cases, the surface fat content is obtained from the body size, the body fat content in the blood and muscle cannot be measured, and the application scenarios are limited. At present, a widely recognized pet obesity measuring method is a BCS scoring system. However, the BCS scoring system has high professional requirements and experience requirements on veterinarians, and is a perceptual evaluation method which is easy to cause evaluation errors.

By aiming at the problems in the related art, there is no effective solution in the prior art.

### SUMMARY

By aiming at the problems in the related art, the present disclosure provides a modular smart body fat scale with a pet body configuration detection function to solve the technical problems in the related art.

Therefore, the present disclosure adopts the following specific technical solution:

A modular smart body fat scale with a pet body configuration detection function includes a main body base and a wireless display. An installing cavity is formed in a front side of the top of the main body base, four weighting sensors arranged at equal intervals are disposed at the inside top of the installing cavity, an installing box is disposed at a top end of the weighting sensor, a matrix grid electrode is disposed inside the installing box, and a cover plate is disposed on the top of the installing box. A support frame bottom groove is formed in the back portion of the top of the main body base, a bottom motor box is disposed in the inside middle portion of the support frame bottom groove, a support upright post is disposed on a top end of the bottom motor box, and a support cross beam is disposed on a top end of the support upright post. A limiting inner slide groove is formed inside each of the support upright post and the support cross beam, a ball screw is disposed inside each limiting inner slide groove, a translational slide block sleeves at the circumference outer side of each ball screw, and a camera component is disposed at one side of each translational slide block.

Further, in order that the installing box and the matrix grid electrode can be stably installed and fixed and protected in a sealed manner by the cover plate, and installing screw bolts achieve a limiting fixation effect without influencing the measuring precision of a weighting sensor by formation of too tight pressure on the installing box, installing grooves are formed at four corners of the top of the installing cavity, installing side lugs matched with the installing grooves are disposed at four corners of the top of the installing box, and an installing screw bolt is disposed between each of the installing grooves and the corresponding installing side lug. A clamping inner frame is disposed at a bottom outer edge of the cover plate and keeps clamping cooperation with the installing box, and pinching grooves are formed at two sides of the top of the cover plate.

Further, in order to form a large-area continuous impedance detection network, the precise calculation on the pet body impedance is achieved by capturing and measuring falling positions of pet palms. The matrix grid electrode is tightly matched with conductive keycaps inside the cover plate to form a complete loop with the conductive substrates, and the smooth operation and the detection precision of the body fat scale are ensured. The matrix grid electrode includes a circuit board disposed at the inside bottom of the installing box, a plurality of electrode posts arranged at equal intervals and distributed to present a rectangular shape are disposed on a top end of the circuit board, a grid junction wire is disposed between the tops of every two adjacent electrode posts, and a magnetic contact is disposed on a top end of each of the electrode posts. A plurality of conductive keycaps arranged at equal intervals and distributed to present a rectangular shape are disposed on the top of the cover plate in a penetrating manner, the conductive keycaps are in one-to-one correspondence to the electrode posts in the quantity and positions, and bottom ends of the conductive keycaps keep connection with the magnetic contacts. A plurality of conductive contacts are disposed at the back portion of the installing box, conductive substrates matched with the conductive contacts are disposed at the back portion of the installing cavity, and the conductive contacts keep electric connection with the circuit board.

Further, in order that the bottom motor box can be limited and fixed through side support plates at two sides to ensure the stability of the bottom motor box in its installing process, a power supply interface at the bottom end is matched with a power supply bottom groove, the stability of the bottom motor box can be further improved, and the power supply and data transmission are kept. Additionally, through magnets, the attraction fixation to the side support plates can be achieved after the bottom motor box is dismounted, and the support frame bottom groove is protected in a sealed manner to a certain degree. Side support plates are disposed at two sides inside the support frame bottom groove and keep rotating shaft connection, an open groove is formed at one side of each of the side support plates near the bottom motor box, side limiting through columns matched with the open grooves are disposed at two sides of a top end of the bottom motor box, and a side limiting screw bolt is disposed between each of the open grooves and the corresponding side limiting through column. A power supply bottom groove is disposed in a center position of an inside bottom end of the support frame bottom groove, and a magnet is disposed at each of two sides of the inside bottom end of the support frame bottom groove and is located between the power supply bottom groove and each of the side support plates. A power supply interface matched with the power supply bottom groove is disposed at a bottom end of the bottom motor box.

Further, in order to achieve stable connection between the support upright post and the support cross beam, ensure the transverse and longitudinal stability of the support cross beam and avoid swinging and side tumbling in a use process, a fixing component is disposed between the top end of the support upright post and the support cross beam. The fixing component includes fixing side plates disposed at two sides of the top end of the support upright post, a fixing inner groove is formed at the inner side of a top end of each of the fixing side plates, and a fixing bottom block is disposed at the outer side of the top end of each of the fixing side plates. A fixing side strip matched with the corresponding fixing inner groove is disposed at each of two sides of the support cross beam, a fixing top rod is disposed at a top end of the support cross beam, and a fixing screw bolt is disposed between the fixing top rod and each of the fixing bottom blocks.

Further, in order to achieve the power supply and data transmission of the top end support beam and inside camera components thereof and achieve an installation limiting effect, a top motor box is disposed at the back portion of the support cross beam, an electrification fixing post is disposed at one side of a bottom end of the top motor box near the support upright post, and an electrification fixing base matched with the electrification fixing post is disposed on the top of the back side of the support upright post.

Further, in order to realize real-time detection videos and photos of a pet and precisely capture and measure the pet body configuration, the cross section of each of the limiting inner slide grooves and the translational slide blocks is of a structure in a shape like a Chinese character “ [symbol] ”. Each of the camera components includes a connecting plate disposed at one side of the corresponding translational slide block near the installing cavity, and a supplementary lighting lamp, a camera and a laser distance sensor are sequentially disposed at a front side of the connecting plate from bottom to top.

Further, in order to enhance the functionality and practicability of the body fat scale and ensure the safe power supply and data transmission and control among all modules, a display screen is disposed at the top of the main body base and is located between the installing cavity and the support frame bottom groove, a control electric box is disposed at the back portion of the main body base, and a core control unit is disposed inside the main body base. Buffer side strips are disposed at two sides and a front side of the main body base. An electronic measuring ruler is disposed at one side of the main body base, and the electronic measuring ruler and the wireless display keep wireless electric connection with the core control unit.

The present disclosure has the beneficial effects:

1. Through arrangement of the matrix grid electrode, a full body length of a pet can be judged through touching positions of pet palms. A smart camera monitoring system is used for cooperation, i.e., machine vision recognition is adopted for BCS scoring on the pet body fat instead of manual operation, additionally, the pet body fat is obtained by a biological resistance method, and pet body fat data is comprehensively given. Errors possibly caused by adopting a single measuring method are overcome, so that precise measurement on the pet body condition is achieved without any invasive injury on the pet, and the health state of the pet is ensured. Additionally, by adding structures such as the weighting sensor, the body fat scale can be integrated with a multifunctional detection system with functions such as weighting, heart rate detection, body fat detection and body configuration detection, and the practicability and functionality of the body fat scale are further greatly improved.

2. Through a modular structure design, the dismounting and mounting difficulty of the body fat scale can be greatly reduced on the premise of ensuring the functionality and stability of the body fat scale, and additionally, the later maintenance and overhaul difficulty and cost of the body fat scale are reduced. That is, the single-module selective installation and repair replacement can be performed according to specific functions, and conditions such as damage to a body fat scale body in a pet feeding process are avoided. Additionally, the connection among all modules is simple and convenient, the data and electric transmission is stable, and the service life of the body fat scale is effectively ensured.

### BRIEF DESCRIPTION OF THE DRAWINGS

In order to describe the technical solution of embodiments of the present disclosure or in the prior art more clearly, drawings to be used in embodiments are briefly introduced. Obviously, drawings described hereafter are only some embodiments of the present disclosure. For a person of ordinary skill in the art, other figures can also be obtained according to these figures without any inventive efforts.

FIG. 1 is a schematic front structure view of a modular smart body fat scale with a pet body configuration detection function according to an embodiment of the present disclosure.

FIG. 2 is a bottom view of a modular smart body fat scale with a pet body configuration detection function according to an embodiment of the present disclosure.

FIG. 3 is an assembly diagram of a matrix grid electrode portion of a modular smart body fat scale with a pet body configuration detection function according to an embodiment of the present disclosure.

FIG. 4 is an assembly diagram of distribution of a support upright post in a matrix grid electrode portion of a modular smart body fat scale with a pet body configuration detection function according to an embodiment of the present disclosure.

FIG. 5 is a side cross-sectional view of a modular smart body fat scale with a pet body configuration detection function according to an embodiment of the present disclosure.

FIG. 6 is an enlarged partial view of a portion A in FIG. 1.

FIG. 7 is an enlarged partial view of a portion B in FIG. 3.

FIG. 8 is an enlarged partial view of a portion C in FIG. 4.

FIG. 9 is an enlarged partial view of a portion D in FIG. 4.

FIG. 10 is an enlarged partial view of a portion E in FIG. 5.

FIG. 11 is a schematic diagram of a voltage equivalent model of a left front palm and a left rear palm of a pet in an embodiment of a modular smart body fat scale with a pet body configuration detection function according to an embodiment of the present disclosure.

FIG. 12 is a schematic diagram of a voltage equivalent model of two rear palms of a pet in an embodiment of a modular smart body fat scale with a pet body configuration detection function according to an embodiment of the present disclosure.

FIG. 13 is a schematic structure diagram of a wireless display in a modular smart body fat scale with a pet body configuration detection function according to an embodiment of the present disclosure.

FIG. 14 is a schematic structure diagram of another embodiment of a modular smart body fat scale with a pet body configuration detection function according to an embodiment of the present disclosure.

FIG. 15 is a structure assembly diagram of another embodiment of a modular smart body fat scale with a pet body configuration detection function according to an embodiment of the present disclosure.

FIG. 16 is a schematic diagram of a structure application process of another embodiment of a modular smart body fat scale with a pet body configuration detection function according to an embodiment of the present disclosure.

FIG. 17 is a schematic diagram of a modular smart body fat scale with a pet body configuration detection function according to an embodiment of the present disclosure provided with an electronic measuring ruler according to another embodiment.

In the figures:
1 denotes a main body base, 2 denotes an installing cavity, 3 denotes a weighting sensor, 4 denotes an installing box, 5 denotes a matrix grid electrode, 501 denotes a circuit board, 502 denotes an electrode post, 503 denotes a grid junction wire, 504 denotes a magnetic contact, 6 denotes a cover plate, 7 denotes a support frame bottom groove, 8 denotes a bottom motor box, 9 denotes a support upright post, 10 denotes a support cross beam, 11 denotes a limiting inner slide groove, 12 denotes a ball screw, 13 denotes a translational slide block, 14 denotes a camera component, 1401 denotes a connecting plate, 1402 denotes a supplementary lighting lamp, 1403 denotes a camera, 1404 denotes a laser distance sensor, 15 denotes an installing groove, 16 denotes an installing side lug, 17 denotes an installing screw bolt, 18 denotes a pinching groove, 19 denotes a conductive keycap, 20 denotes a conductive contact, 21 denotes a conductive substrate, 22 denotes a side support plate, 23 denotes an open groove, 24 denotes a side limiting through column, 25 denotes a side limiting screw bolt, 26 denotes a power supply bottom groove, 27 denotes a magnet, 28 denotes a power supply interface, 29 denotes a fixing component, 2901 denotes a fixing side plate, 2902 denotes a fixing inner groove, 2903 denotes a fixing bottom block, 2904 denotes a fixing side strip, 2905 denotes a fixing top rod, 2906 denotes a fixing screw bolt, 30 denotes a top motor box, 31 denotes an electrification fixing post, 32 denotes an electrification fixing base, 33 denotes a display screen, 34 denotes a control electric box, 35 denotes a core control unit, 36 denotes a buffer side strip, 37 denotes a wireless display, and 38 denotes an electronic measuring ruler.

### DETAILED DESCRIPTION OF THE DISCLOSURE

In order to further illustrate each embodiment, the present disclosure provides accompanying drawings, which constitute a part of the present disclosure, are mainly used for illustrating the embodiments and can be used to explain operation principles of the embodiments together with relevant description contents of the Description. Other possible embodiments and advantages of the present disclosure will be readily apparent to those of ordinary skill in the art from these contents. Components in the figures are not drawn to the scale, and like reference numerals are generally used to refer to like components.

According to an embodiment of the present disclosure, a modular smart body fat scale with a pet body configuration detection function is provided.

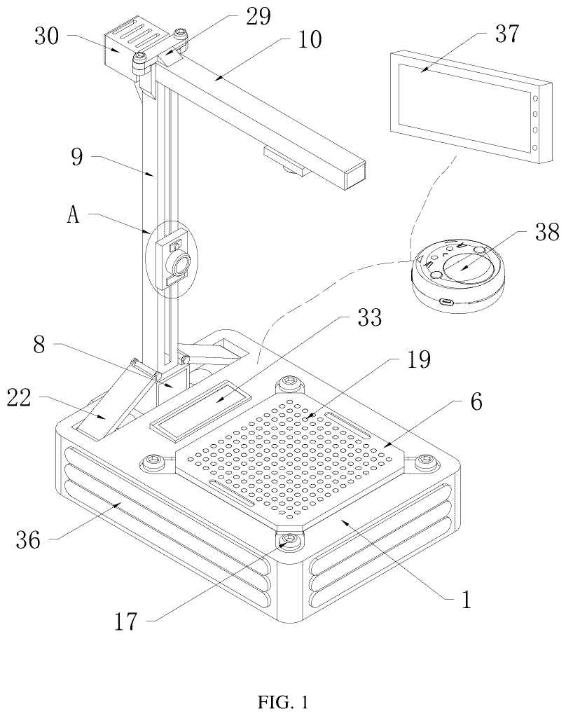

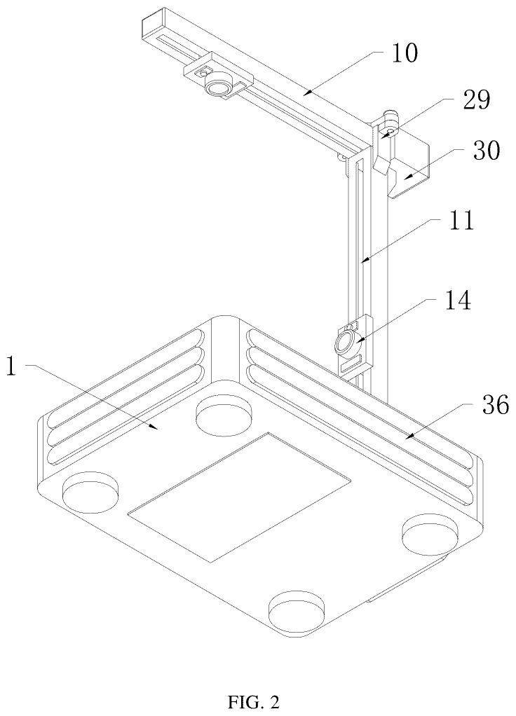

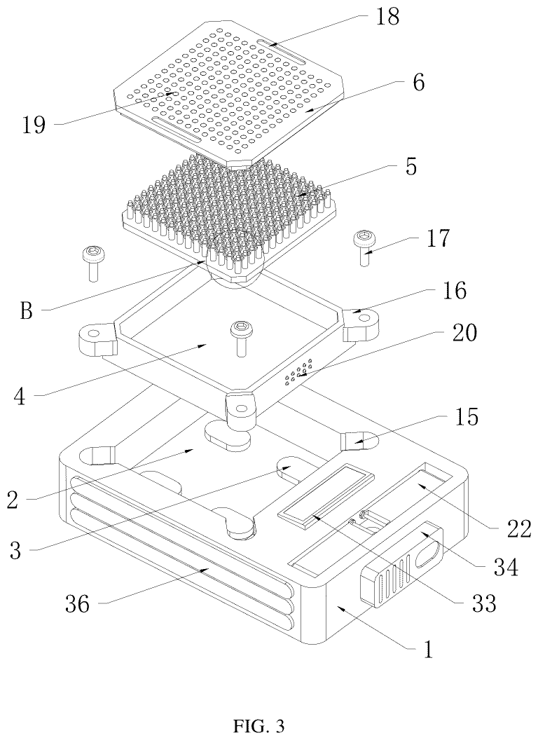

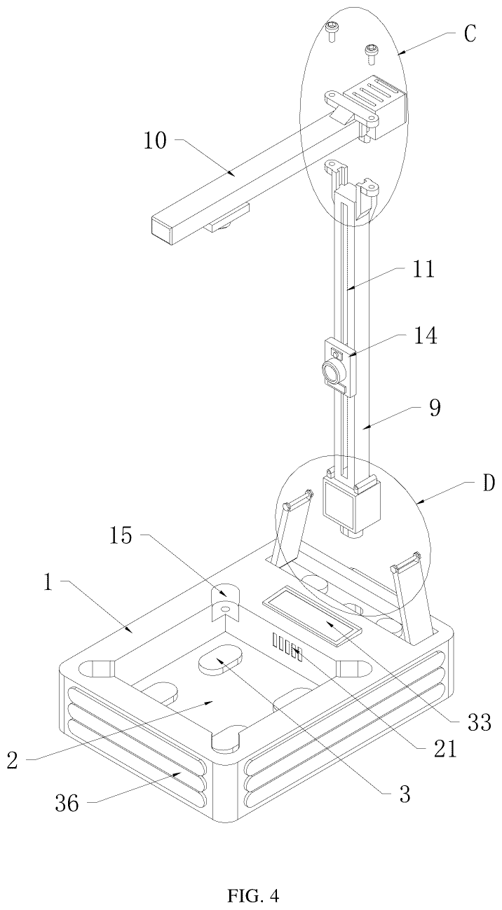

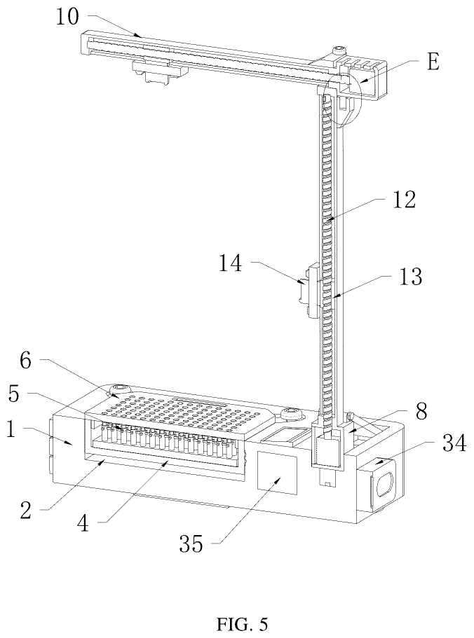

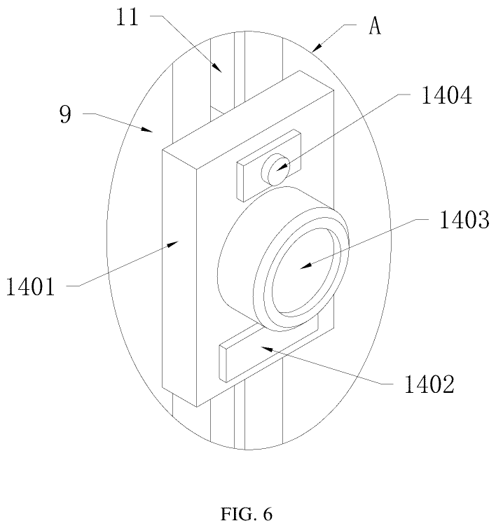

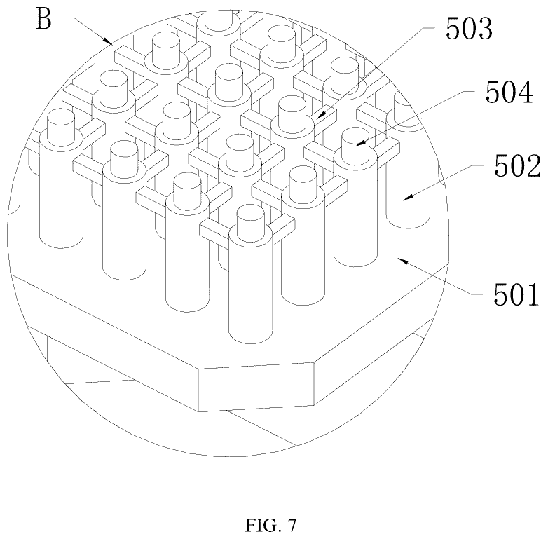

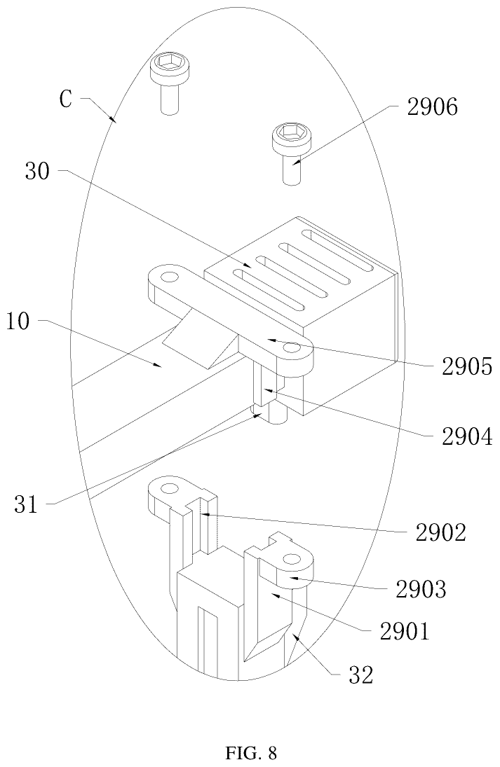

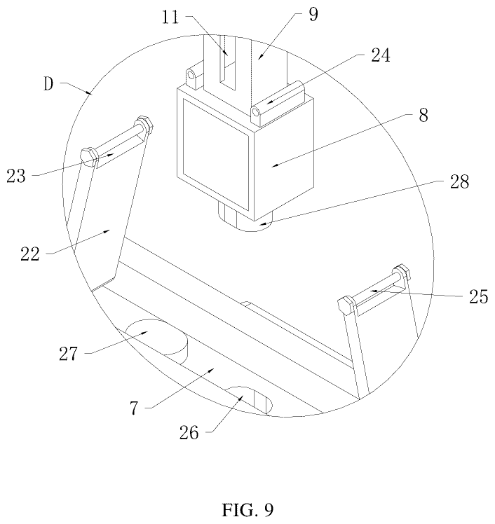

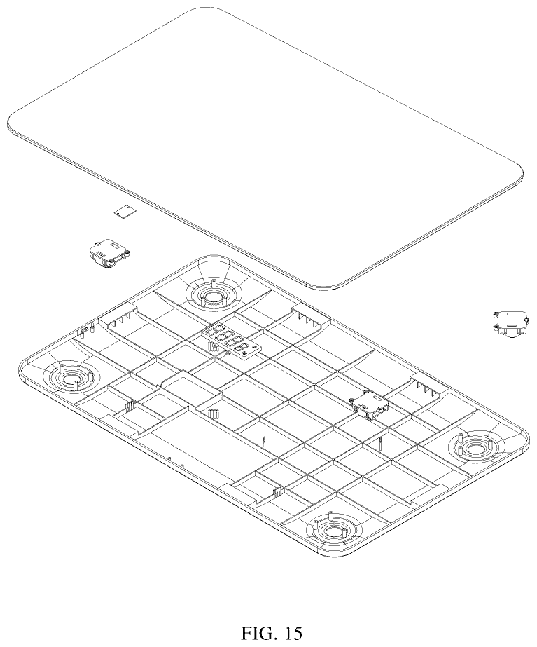

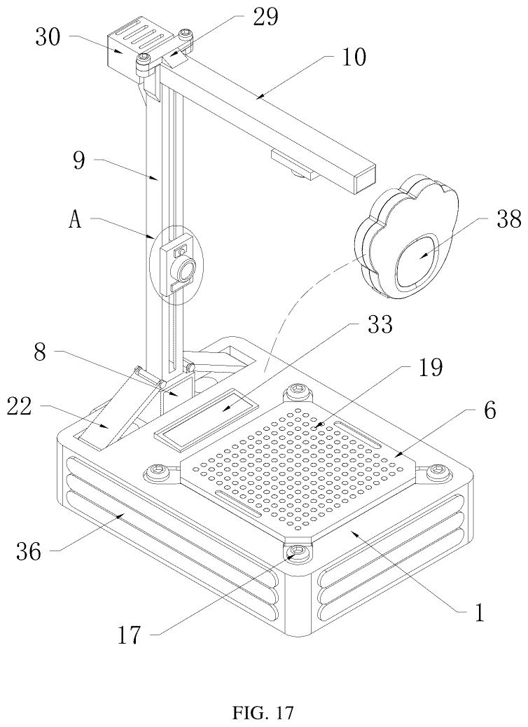

The present disclosure will be further illustrated in conjunction with the drawings and embodiments. As shown in FIG. 1 to FIG. 17, a modular smart body fat scale with a pet body configuration detection function according to an embodiment of the present disclosure includes a main body base 1 and a wireless display 37. An installing cavity 2 is formed in a front side of the top of the main body base 1, four weighting sensors 3 arranged at equal intervals are disposed at the inside top of the installing cavity 2, an installing box 4 is disposed at a top end of the weighting sensor 3, a matrix grid electrode 5 is disposed inside the installing box 4, and a cover plate 6 is disposed on the top of the installing box 4. A support frame bottom groove 7 is formed in the back portion of the top of the main body base 1, a bottom motor box 8 is disposed in the inside middle portion of the support frame bottom groove 7, a support upright post 9 is disposed on a top end of the bottom motor box 8, and a support cross beam 10 is disposed on a top end of the support upright post 9. A limiting inner slide groove 11 is formed inside each of the support upright post 9 and the support cross beam 10, a ball screw 12 is disposed inside each limiting inner slide groove 11, a translational slide block 13 sleeves at the circumference outer side of each ball screw 12, and a camera component 14 is disposed at one side of each translational slide block 13.

By adopting the technical solution, through arrangement of the matrix grid electrode 5, a full body length of a pet can be judged through touching positions of pet palms. A smart camera monitoring system is used for cooperation, i.e., machine vision recognition is adopted for BCS scoring on the pet body fat instead of manual operation, additionally, the pet body fat is obtained by a biological resistance method, and pet body fat data is comprehensively given. Errors possibly caused by adopting a single measuring method are overcome, so that precise measurement on the pet body condition is achieved without any invasive injury on the pet, and the health state of the pet is ensured. Additionally, by adding structures such as the weighting sensor, the body fat scale can be integrated with a multifunctional detection system with functions such as weighting, heart rate detection, body fat detection and body configuration detection, and the practicability and functionality of the body fat scale are further greatly improved. Through a modular structure design, the dismounting and mounting difficulty of the body fat scale can be greatly reduced on the premise of ensuring the functionality and stability of the body fat scale, and additionally, the later maintenance and overhaul difficulty and cost of the body fat scale are reduced. That is, the single-module selective installation and repair replacement can be performed according to specific functions, and conditions such as damage to a body fat scale body in a pet feeding process are avoided. Additionally, the connection among all modules is simple and convenient, the data and electric transmission is stable, and the service life of the body fat scale is effectively ensured.

In an embodiment, for the installing cavity 2, installing grooves 15 are formed at four corners of the top of the installing cavity 2, installing side lugs 16 matched with the installing grooves 15 are disposed at four corners of the top of the installing box 4, and an installing screw bolt 17 is disposed between each of the installing grooves 15 and the corresponding installing side lug 16. A clamping inner frame is disposed at a bottom outer edge of the cover plate 6 and keeps clamping cooperation with the installing box 4, and pinching grooves 18 are formed at two sides of the top of the cover plate 6, so that the installing box 4 and the matrix grid electrode 5 can be stably installed and fixed and protected in a sealed manner by the cover plate 6. The installing screw bolts 17 achieve a limiting fixation effect without influencing the measuring precision of a weighting sensor 3 by formation of too tight pressure on the installing box 4.

In an embodiment, for the matrix grid electrode 5, the matrix grid electrode 5 includes a circuit board 501 disposed at the inside bottom of the installing box 4, a plurality of electrode posts 502 arranged at equal intervals and distributed to present a rectangular shape are disposed on a top end of the circuit board 501, a grid junction wire 503 is disposed between the tops of every two adjacent electrode posts 502, and a magnetic contact 504 is disposed on a top end of each of the electrode posts 502. A plurality of conductive keycaps 19 arranged at equal intervals and distributed to present a rectangular shape are disposed on the top of the cover plate 6 in a penetrating manner, the conductive keycaps 19 are in one-to-one correspondence to the electrode posts 502 in the quantity and positions, and bottom ends of the conductive keycaps 19 keep connection with the magnetic contacts 504. A plurality of conductive contacts 20 are disposed at the back portion of the installing box 4, conductive substrates 21 matched with the conductive contacts 20 are disposed at the back portion of the installing cavity 2, and the conductive contacts 20 keep electric connection with the circuit board 501, so that a large-area continuous impedance detection network can be formed. The precise calculation on the pet body impedance is achieved by capturing and measuring falling positions of pet palms. The matrix grid electrode 5 is tightly matched with the conductive keycaps 19 inside the cover plate 6 to form a complete loop with the conductive substrates 21, and the smooth operation and the detection precision of the body fat scale are ensured.

In an embodiment, for the support frame bottom groove 7, side support plates 22 are disposed at two sides inside the support frame bottom groove 7 and keep rotating shaft connection, an open groove 23 is formed at one side of each of the side support plates 22 near the bottom motor box 8, side limiting through columns 24 matched with the open grooves 23 are disposed at two sides of a top end of the bottom motor box 8, and a side limiting screw bolt 25 is disposed between each of the open grooves 23 and the corresponding side limiting through column 24. A power supply bottom groove 26 is disposed in a center position of an inside bottom end of the support frame bottom groove 7, and a magnet 27 is disposed at each of two sides of the inside bottom end of the support frame bottom groove 7 and is located between the power supply bottom groove 26 and each of the side support plates 22. A power supply interface 28 matched with the power supply bottom groove 26 is disposed at a bottom end of the bottom motor box 8, so that the bottom motor box 8 can be limited and fixed through the side support plates 22 at two sides to ensure the stability of the bottom motor box in its installing process. The power supply interface 28 at the bottom end is matched with the power supply bottom groove 26, the stability of the bottom motor box 8 can be further improved, and the power supply and data transmission are kept. Additionally, through the magnets 27, the attraction fixation to the side support plates 22 can be achieved after the bottom motor box 8 is dismounted, and the support frame bottom groove 7 is protected in a sealed manner to a certain degree.

In an embodiment, for the support upright post 9, a fixing component 29 is disposed between the top end of the support upright post 9 and the support cross beam 10. The fixing component 29 includes fixing side plates 2901 disposed at two sides of the top end of the support upright post 9, a fixing inner groove 2902 is formed at the inner side of a top end of each of the fixing side plates 2901, and a fixing bottom block 2903 is disposed at the outer side of the top end of each of the fixing side plates 2901. A fixing side strip 2904 matched with the corresponding fixing inner groove 2902 is disposed at each of two sides of the support cross beam 10, a fixing top rod 2905 is disposed at a top end of the support cross beam 10, and a fixing screw bolt 2906 is disposed between the fixing top rod 2905 and each of the fixing bottom blocks 2903, so that the stable connection between the support upright post 9 and the support cross beam 10 can be achieved, the transverse and longitudinal stability of the support cross beam 10 are ensured, and swinging and side tumbling in a use process are avoided.

In an embodiment, for the support cross beam 10, a top motor box 30 is disposed at the back portion of the support cross beam 10, an electrification fixing post 31 is disposed at one side of a bottom end of the top motor box 30 near the support upright post 9, and an electrification fixing base 32 matched with the electrification fixing post 31 is disposed on the top of the back side of the support upright post 9, so that the power supply and data transmission of the top end support beam 10 and inside camera components 14 thereof can be achieved, and an installation limiting effect can be achieved.

In an embodiment, for the limiting inner slide grooves 11, the cross section of each of the limiting inner slide grooves 11 and the translational slide blocks 13 is of a structure in a shape like a Chinese character “ [symbol] ”. Each of the camera components 14 includes a connecting plate 1401 disposed at one side of the corresponding translational slide block 13 near the installing cavity 2, and a supplementary lighting lamp 1402, a camera 1403 and a laser distance sensor 1404 are sequentially disposed at a front side of the connecting plate 1401 from bottom to top, so that real-time detection videos and photos of a pet can be realized, and the pet body configuration are precisely captured and measured.

In an embodiment, for the main body base 1, a display screen 33 is disposed at the top of the main body base 1 and is located between the installing cavity 2 and the support frame bottom groove 7, a control electric box 34 is disposed at the back portion of the main body base 1, and a core control unit 35 is disposed inside the main body base 1. Buffer side strips 36 are disposed at two sides and a front side of the main body base 1. An electronic measuring ruler 38 is disposed at one side of the main body base 1, and the electronic measuring ruler 38 and the wireless display 37 keep wireless electric connection with the core control unit 35, so that the functionality and practicability of the body fat scale can be enhanced, and the safe power supply and data transmission and control among all modules can be ensured.

In order to conveniently understand the technical solution of the present disclosure, working principles or operation manners of the present disclosure in a practical use process are illustrated hereafter in detail.

In practical application, a user assembles the body fat scale in advance. That is, the installing box 4 with the matrix grid electrode 5 is firstly installed inside the installing cavity 2. In a process of fixing the installing screw bolts 17, a downward pressure is withdrawn after the installed box 4 cannot fall off, so that the measuring precision of the weighting sensor 3 cannot be influenced, and the weight of the installing box 4 and inside weight thereof will return to zero at first in a practical weighting process. After the installation is completed, the side support plates 22 are opened to two sides, the bottom motor box 8 is inserted into the support frame bottom groove 7. Until the power supply interface 28 is inserted into the power supply bottom groove 26, the side support plates 22 at the two sides are turned to the outer sides of the side limiting through columns 24, and the side limiting screw bolts 25 are taken down to be inserted into the coaxial holes to complete limiting fixation. Finally, the support cross beam 10 is installed on the top end of the support upright post 9.

After connection with a power supply, a pet is placed to the top end of the cover plate 6, the pet can be weighted and measured. In the measuring process, the body fat scale is connected with an upper computer or a computer through an inside WiFi module and is used to monitor photo data and measuring data in real time and perform real-time adjustment control.

An impedance detection principle of the matrix grid electrode 5 is as follows: supposed that a point A and a point B at the top end of the cover plate 6 are communicated, at this moment, an alternating current constant current source (direct current may cause polarization, like charging to a capacitor, the current is weaker as the time goes, and the present disclosure adopts an alternating current constant current source 60 KHz/500 uA for electrolyte measurement) is input between the point A and the point B, and is recorded as Eab. A sampling voltage between the point A and the point B is recorded as Vab. Treatment such as rectification filtering is performed, then, a measured analog signal is converted into a digital signal through ADC, so that a voltage value of the body impedance is acquired. Since the current is certain, the body impedance can be obtained through calculation. By using a bioelectrical impedance principle, a body fat content value can be obtained through calculation.

In a practical measuring process on a pet, it can be divided into two solutions to measure the pet body impedance.

### Measurement Solution 1:

Firstly, a standing position of the pet is judged, Vab is recorded as 1 when being a voltage threshold value of current flowing through palms, and otherwise, Vab is zero. Supposed that four regions on the cover plate 6 are in contact with the four palms of the pet, the four regions are marked as standing random positions of the four palms of the pet. A process is as follows:

$$A — S 1 -- SA - 1 -- palm — SB - 1 — S 11 — B \qquad V ab = 0$$

$$A — S 2 -- SA - 2 -- palm — SB - 2 — S 12 — B \qquad V ab = 1$$

$$A — S 3 -- SA - 3 -- palm — SB - 3 — S 13 — B \qquad V ab = 1$$

The above process is repeated to sequentially completing judgment of the whole electrode:

$$A — S 5 -- SA - 5 -- palm — SB - 5 — S 15 — B \qquad V ab = 1$$

$$A — S 6 -- SA - 6 -- palm — SB - 6 — S 16 — B \qquad V ab = 1$$

$$A — S 1 -- SF - 1 -- palm — SG - 1 — S 11 — B \qquad V ab = 1$$

$$A — S 2 -- SF - 2 -- palm — SG - 1 — S 12 — B \qquad V ab = 1$$

$$A — S 4 -- SG - 4 -- palm — SH - 4 — S 14 — B \qquad V ab = 1$$

$$A — S 5 -- SG - 5 -- palm — SH - 5 — S 15 — B \qquad V ab = 1$$

According to the position of Vab=1, the practical standing positions of the palms on the top end of the cover plate 6 can be judged, and measurement at two sides can be performed after the position information is determined:

During first measurement, the impedance is recorded as Z1, and Zllrc1 and Zllrc2 are respectively contact resistance between the left front and rear palms and the electrode.

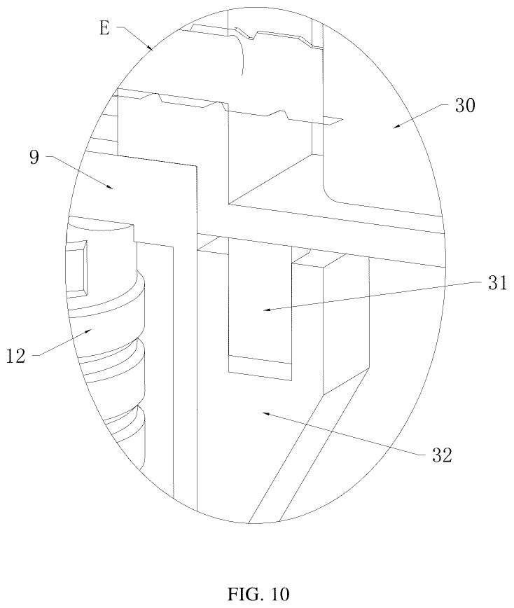

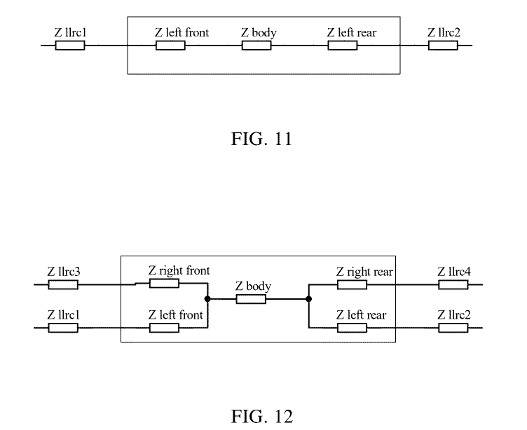

The current is enabled to flow into the body from the left front palm and flow out of the body from the left rear palm, a measured voltage is recorded as Vab1, the impedance Z1 is obtained through calculation by using Vab1, and an equivalent model is as shown in FIG. 11.

During second measurement, the impedance is recorded as Z2, and Zllrc3 and Zllrc4 are respectively contact resistance between the right front and rear palms and the electrode.

The current is enabled to flow into the body from the two front palms and flow out of the body from the two rear palms, a measured voltage is recorded as Vab2, the impedance Z2 is obtained through calculation by using Vab2, and an equivalent model is as shown in FIG. 12.

The total body impedance is as follows: Z=2Z2−Z1.

A body size of the pet can be approximately judged through electrode plates in a position of Vab=1, and the approximate size of the pet can be judged through the single palm conduction quantity.

### Measurement Solution 2:

In the process of measuring by the measurement solution1, if there is a certain deviation between the two sides of the pet's body, there will be a certain measurement error. To solve this problem, measurement solution 2 has been improved from the solution 1. The specific solution is as follows:
(1) Combine the front palms and pour current, and then flow Z2 out of the back palms;
(2) The current is enabled to flow into the body from the left front palm and flow out of the body from the left rear palm, measured the impedance Z1;
(3) The current is enabled to flow into the body from the right front palm and flow out of the body from the right rear palm, measured the impedance Z11;
(4) The current is enabled to flow into the body from the left front palm and flow out of the body from the right rear palm, measured the impedance Z12;
(5) The current is enabled to flow into the body from the right front palm and flow out of the body from the left rear palm, measured the impedance Z13;
The result of the first measurement is Z2. Multiply the result by 2 to get 2Z2; The second and third measurement results are in parallel, the third and fourth measurement results are in parallel, and the above two results are added and recorded as Za.

Then the body impedance is as follows:

$$Zbody = 2 Z 2 - ( Z 1 // Z 11 + Z 12 // Z 13 ) = 2 Z 2 - Za .$$

The pet size measurement may also be performed through an external electronic measuring ruler 38 (including two measuring ruler types). For multiple-circumference measurement of the pet, a traditional method is to use an ordinary tape measure in the market for measuring. When this method is used, a professional person is needed during measurement, or the size data of each circumference of the pet can be accurately obtained through measurement by a person after being trained by a professional person. Since the result obtained through estimation by eyes of a measuring person cannot be very accurate, errors are unavoidable. Additionally, errors by mistake may also be caused when the measuring person is careless during measuring result reading and recording. By aiming at this defect, the present disclosure adopts and manufactures a flexible electronic measuring ruler 38. A capacitance encoder has the characteristics of high impedance and low power, so the capacitance encoder is used to be connected with a flexible soft ruler. The capacitance encoder can detect an out-extending length of the flexible ruler, then, the data is displayed through a display screen, and at the same time, the data can be sent to an upper computer through Bluetooth built in the electronic ruler. A specific use flow process of the upper computer is as follows:
1. An upper computer program (mobile phone application program or desktop program) is started to enter a pet size measuring interface. A head circumference, a neck circumference, an upper torso circumference, a lower torso circumference, a full torso length and a full body length may be filled to the size measuring interface.
2. The ruler is started, the Bluetooth connection with the ruler may be realized, and numbers on a ruler screen may be automatically input into a textbox of the measuring interface.
3. When a built-in press key of the ruler is touched and held, the unit of the ruler may be switched, the measuring interface may be accordingly changed, and additionally, previous measured values may be changed into values corresponding to the current unit.
4. When the built-in press key on the ruler is clicked, an input cursor of the measuring interface may automatically jump to a next position. When the input cursor is in a last position, the size will be uploaded to the cloud.
5. A body fat value of the pet can be obtained through calculation by combining the measured pet size with the body fat scale.

Additionally, the present disclosure adopts machine vision recognition for BCS scoring on the pet body fat instead of manual operation, additionally, the pet body fat is obtained by a biological resistance method, and pet body fat data is comprehensively given. Errors possibly caused by adopting a single measuring method are overcome. In this process, the camera components 14 and the ball screws 12 are operated and controlled to automatically run.

The cameras 1403 are based on an electromagnetic wave imaging technology, photo taking on a real contour of the pet without hairs can be realized, shot photos are uploaded to the upper computer, the upper computer compares a treated pet contour with a pet BCS standard contour by adopting an OpenCV video contour matching algorithm, a contour the same as the standard contour is selected out, and a BCS score of the pet can be obtained.

The laser distance sensor 1404 is configured to acquire a distance from the pet to the camera, and then, the measurement on the size of each portion of the pet can be realized by combining with the photo obtained from the camera.

The ball screw 12 is used inside the support upright post 9 for pushing, the ball screw 12 is driven by a step motor, and automatic control is realized. The pet contour is detected through the laser distance sensor 1404 and the camera 1403, the middle position of the pet body is found, and a coordinate of the middle is sent to the step motor, so that the step motor drives the ball screw 12, the camera 1403 reaches the middle position of the pet body, and a relatively standard pet contour photo is obtained.

Work flow process of a pet vision recognition system:
1. Program initialization is performed.
2. A height adjusting ball screw 12 return to an origin point (the origin point is located at the upper side).
3. The ball screw 12 pushes the camera 1403 to move down.
4. The ball screw 12 moves down to trigger a short-distance signal (a lead screw moving distance a1 is recorded).
5. The ball screw 12 continuously moves down.
6. A ball screw 12 moves down to trigger a long-distance signal (a lead screw moving distance a2 is recorded).
7. The camera moves to a pet body center position (a1+(a2−a1)/2).
8. The camera 1403 captures a video contour.
9. A contour the same as a standard contour and a target contour are selected.
10. A pet BCS score is output.

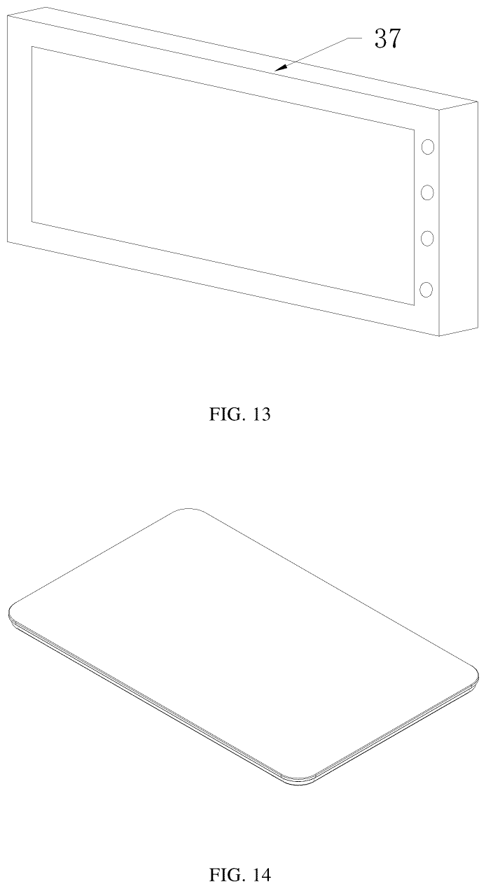

Additionally, as shown in FIG. 13, the wireless display 37 adopts a dismountable design independent from the body fat scale, can be externally hung on a wall or placed on a desktop. The wireless display 37 has a WiFi module and a Bluetooth function, a clock and a weight value measured by the body fat scale can be displayed, a real-time display function is achieved, and better humanization is achieved.

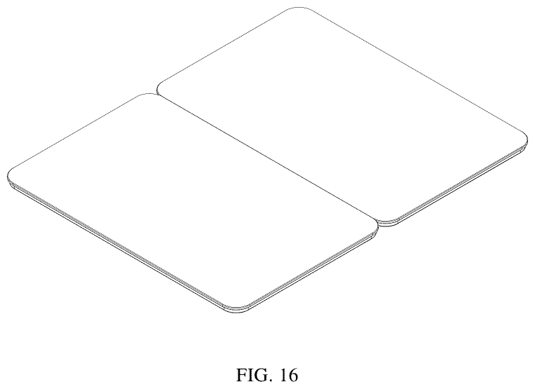

In another embodiment of the present disclosure, the core function of the body fat scale per se is pet body fat and body weight measurement. Therefore, a combined modular structure disclosed by the present disclosure can realize independent configuration according to the specific application scenario. For example, the specific body fat value of the pet is measured by only utilizing a matrix electrode principle. As shown in FIG. 14 to FIG. 16, the body fat scale is provided with a top plate formed by the matrix grid electrode disclosed by the present disclosure. The functions of the matrix grid electrode 5 and the cover plate 6 are integrated, and thin appearance and simplified structure are realized. That is, a screw bolt connection manner is avoided, and clamp buckle connection installation is adopted. However, an operation manner and a detection principle are the same as those described in the embodiments. Through cooperation with a customized base at the bottom, the cover plate with the matrix grid electrode can be installed and stored. Additionally, through the weighting sensors disposed at four corners, the measurement of the body weight is realized. Therefore, the body weight scale, used as a simplified structure with partial body fat measuring functions in the present disclosure, can independently run to be used, and the practicability is further greatly improved. In a use process, in order to ensure the detection precision and effect, a manner of splicing two body fat scales for use is generally adopted. As shown in FIG. 16, even if front and rear palms of the pet respectively stand on the two body fat scales, information such as the pet body fat rate can be judged through the current change of the matrix grid electrodes of the two body fat scales, and the detection precision is effectively improved.

Based on the above, by adopting the technical solution of the prevent disclosure, through the arrangement of the matrix grid electrode 5, the full body length of the pet can be judged through the position touched by the pet palms. Through the cooperation with a smart camera monitoring system, the machine vision recognition is adopted for BCS scoring on the pet body fat instead of manual operation, additionally, the pet body fat is obtained by a biological resistance method, and pet body fat data is comprehensively given. Errors possibly caused by adopting a single measuring method are overcome, so that precise measurement on the pet body condition is achieved without any invasive injury on the pet, and the health state of the pet is ensured. Additionally, by adding structures such as the weighting sensor, the body fat scale can be integrated with a multifunctional detection system with functions such as weighting, heart rate detection, body fat detection and body configuration detection, and the practicability and functionality of the body fat scale are further greatly improved. Through a modular structure design, the dismounting and mounting difficulty of the body fat scale can be greatly reduced on the premise of ensuring the functionality and stability of the body fat scale, and additionally, the later maintenance and overhaul difficulty and cost of the body fat scale are reduced. That is, the single-module selective installation and repair replacement can be performed according to specific functions, and conditions such as damage to a body fat scale body in a pet feeding process are avoided. Additionally, the connection among all modules is simple and convenient, the data and electric transmission is stable, and the service life of the body fat scale is effectively ensured.

In the present disclosure, unless expressly specified or limited otherwise, the terms “installed”, “disposed”, “connected”, “fixed”, “screwed” and the like are to be construed broadly, for example, they may be fixed connection, detachable connection, or integral connection, may be mechanical connection or electrical connection, may be direct connection, may be indirect connection through an intermediate medium, may be communication of inner sides of two elements, or may be interaction between two elements. Unless expressly limited otherwise, the specific meaning of the above terms in the present disclosure can be understood by those of ordinary skill in the art according to specific circumstances.

The foregoing descriptions are merely exemplary embodiments of the present disclosure, but are not intended to limit the present disclosure. Any modification, equivalent substitution, improvement and the like made within the spirit and principle of the present disclosure shall fall within the protection scope of the present disclosure.

1. A modular smart body fat scale with a pet body configuration detection function, comprising a main body base (1) and a wireless display (37), wherein
an installing cavity (2) is formed in a front side of the top of the main body base (1), four weighting sensors (3) arranged at equal intervals are disposed at the inside top of the installing cavity (2), an installing box (4) is disposed at a top end of the weighting sensor (3), a matrix grid electrode (5) is disposed inside the installing box (4), and a cover plate (6) is disposed on the top of the installing box (4);
a support frame bottom groove (7) is formed in the back portion of the top of the main body base (1), a bottom motor box (8) is disposed in the inside middle portion of the support frame bottom groove (7), a support upright post (9) is disposed on a top end of the bottom motor box (8), and a support cross beam (10) is disposed on a top end of the support upright post (9); and
a limiting inner slide groove (11) is formed inside each of the support upright post (9) and the support cross beam (10), a ball screw (12) is disposed inside each limiting inner slide groove (11), a translational slide block (13) sleeves at the circumference outer side of each ball screw (12), and a camera component (14) is disposed at one side of each translational slide block (13).

2. The modular smart body fat scale with a pet body configuration detection function according to claim 1, wherein installing grooves (15) are formed at four corners of the top of the installing cavity (2), installing side lugs (16) matched with the installing grooves (15) are disposed at four corners of the top of the installing box (4), and an installing screw bolt (17) is disposed between each of the installing grooves (15) and the corresponding installing side lug (16); and
a clamping inner frame is disposed at a bottom outer edge of the cover plate (6) and keeps clamping cooperation with the installing box (4), and pinching grooves (18) are formed at two sides of the top of the cover plate (6).

3. The modular smart body fat scale with a pet body configuration detection function according to claim 1, wherein the matrix grid electrode (5) comprises a circuit board (501) disposed at the inside bottom of the installing box (4), a plurality of electrode posts (502) arranged at equal intervals and distributed to present a rectangular shape are disposed on a top end of the circuit board (501), a grid junction wire (503) is disposed between the tops of every two adjacent electrode posts (502), and a magnetic contact (504) is disposed on a top end of each of the electrode posts (502).

4. The modular smart body fat scale with a pet body configuration detection function according to claim 3, wherein a plurality of conductive keycaps (19) arranged at equal intervals and distributed to present a rectangular shape are disposed on the top of the cover plate (6) in a penetrating manner, the conductive keycaps (19) are in one-to-one correspondence to the electrode posts (502) in the quantity and positions, and bottom ends of the conductive keycaps (19) keep connection with the magnetic contacts (504); and
a plurality of conductive contacts (20) are disposed at the back portion of the installing box (4), conductive substrates (21) matched with the conductive contacts (20) are disposed at the back portion of the installing cavity (2), and the conductive contacts (20) keep electric connection with the circuit board (501).

5. The modular smart body fat scale with a pet body configuration detection function according to claim 1, wherein side support plates (22) are disposed at two sides inside the support frame bottom groove (7) and keep rotating shaft connection, an open groove (23) is formed at one side of each of the side support plates (22) near the bottom motor box (8), side limiting through columns (24) matched with the open grooves (23) are disposed at two sides of a top end of the bottom motor box (8), and a side limiting screw bolt (25) is disposed between each of the open grooves (23) and the corresponding side limiting through column (24).

6. The modular smart body fat scale with a pet body configuration detection function according to claim 5, wherein a power supply bottom groove (26) is disposed in a center position of an inside bottom end of the support frame bottom groove (7), and a magnet (27) is disposed at each of two sides of the inside bottom end of the support frame bottom groove (7) and is located between the power supply bottom groove (26) and each of the side support plates (22); and
a power supply interface (28) matched with the power supply bottom groove (26) is disposed at a bottom end of the bottom motor box (8).

7. The modular smart body fat scale with a pet body configuration detection function according to claim 1, wherein a fixing component (29) is disposed between the top end of the support upright post (9) and the support cross beam (10);
the fixing component (29) comprises fixing side plates (2901) disposed at two sides of the top end of the support upright post (9), a fixing inner groove (2902) is formed at the inner side of a top end of each of the fixing side plates (2901), and a fixing bottom block (2903) is disposed at the outer side of the top end of each of the fixing side plates (2901); and
a fixing side strip (2904) matched with the corresponding fixing inner groove (2902) is disposed at each of two sides of the support cross beam (10), a fixing top rod (2905) is disposed at a top end of the support cross beam (10), and a fixing screw bolt (2906) is disposed between the fixing top rod (2905) and each of the fixing bottom blocks (2903).

8. The modular smart body fat scale with a pet body configuration detection function according to claim 1, wherein a top motor box (30) is disposed at the back portion of the support cross beam (10), an electrification fixing post (31) is disposed at one side of a bottom end of the top motor box (30) near the support upright post (9), and an electrification fixing base (32) matched with the electrification fixing post (31) is disposed on the top of the back side of the support upright post (9).

9. The modular smart body fat scale with a pet body configuration detection function according to claim 1, wherein the cross section of each of the limiting inner slide grooves (11) and the translational slide blocks (13) is of a structure in a shape like a Chinese character “H”; and
each of the camera components (14) comprises a connecting plate (1401) disposed at one side of the corresponding translational slide block (13) near the installing cavity (2), and a supplementary lighting lamp (1402), a camera (1403) and a laser distance sensor (1404) are sequentially disposed at a front side of the connecting plate (1401) from bottom to top.

10. The modular smart body fat scale with a pet body configuration detection function according to claim 1, wherein a display screen (33) is disposed at the top of the main body base (1) and is located between the installing cavity (2) and the support frame bottom groove (7), a control electric box (34) is disposed at the back portion of the main body base (1), and a core control unit (35) is disposed inside the main body base (1);
buffer side strips (36) are disposed at two sides and a front side of the main body base (1); and
an electronic measuring ruler (38) is disposed at one side of the main body base (1), and the electronic measuring ruler (38) and the wireless display (37) keep wireless electric connection with the core control unit (35).

11. The modular smart body fat scale with a pet body configuration detection function according to claim 2, wherein the matrix grid electrode (5) comprises a circuit board (501) disposed at the inside bottom of the installing box (4), a plurality of electrode posts (502) arranged at equal intervals and distributed to present a rectangular shape are disposed on a top end of the circuit board (501), a grid junction wire (503) is disposed between the tops of every two adjacent electrode posts (502), and a magnetic contact (504) is disposed on a top end of each of the electrode posts (502).
